SEQUENCE LISTING (8 sequences)

SEQ ID NO: 1 - protein, 330 aa, Homo sapiens [sequence omitted]

SEQ ID NO: 2 - protein, 329 aa, Bos taurus
ASTTAPKVYPLSSCCGDKSSSTVTLGCLVSSYMPEPVTVTWNSGALKSGVHTFPAVLQSSGLYSLSSMVTVPGSTSGTQTFTCNVAHPASSTKVDKAVDPRCKTTCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADGSYFLYSRLRVDRNSWQEGDTYTCVVMHEALHNHYTQKSTSKSAGK

SEQ ID NO: 3 - protein, 308 aa, Ovis aries
TLGCLVSSYMPEPVTVTWNSGALTSGVHTFPAILQSSGLYSLSSVVTVPASTSGAQTFICNVAHPASSTKVDKRVEPGCPDPCKHCRCPPPELPGGPSVFIFPPKPKDTLTISGTPEVTCVVVDVGQDDPEVQFSWFVDNVEVRTARTKPREEQFNSTFRVVSALPIQHQDWTGGKEFKCKVHNEALPAPIVRTISRTKGQAREPQVYVLAPPQEELSKSTLSVTCLVTGFYPDYIAVEWQKNGQPESEDKYGTTTSQLDADGSYFLYSRLRVDKNSWQEGDTYACVVMHEALHNHYTQKSISKPPGK

SEQ ID NO: 4 - protein, 336 aa, Equus caballus
  VARIANT 250: Xaa = Any Amino Acid
ASTTAPKVFPLASHSAATSGSTVALGCLVSSYFPEPVTVSWNSGALTSGVHTFPSVLQSSGLYSLSSMVTVPASSLKSQTYICNVAHPASSTKVDKKIHLSVLSAVIKECGGCPTCPPECLSVGPSVFIFPPKPKDVLMISRTPTVTCVVVDVGHDFPDVQFNWYVDGVETHTATTEPKQEQNNSTYRVVSILAIQHKDWLSGKEFKCKVNNQALPAPVQKTISKPTGQPREPQVYVLAPHRAELSKNKXSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 5 - protein, 324 aa, Mus musculus
AKTTPPSVYPLAPGSAAQTNSMVTLGCLVKGYFPEPVTVTWNSGSLSSGVHTFPAVLESDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 6 - protein, 327 aa, Monodelphis domestica
ASPTAPSVFALAPNCGQGTSSQVAMACLVSNYFPEPVTVTWNSGAISSGIQTYPSILQSSGLYTSSSQLTVPADDWLTKSYICNVAHKPTSTKTDKKIEKISECTCCKCQACDVVGPSVFLFPPNPKDTLTLSRVPKITCVVVDVSDASEVQISWYKGENAIDSPKPTERKLNNGTFQVVSTLSVAHQEWLNGVAYTCKVDNKELPYPERKTIFHTKGNRKKPDVYVFAPHPDELKQKDTVSITCLVKSFFPKEVVVEWQCNNNPESEDNYSTTEAMRENDTFFVYSKLNVKKTKWQENNHYTCTVLHEALPNQTSQRTISASSPGK

SEQ ID NO: 7 - protein, 478 aa, Listeria monocytogenes
DASAFNKEEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 8 - protein, 478 aa, Listeria monocytogenes
DASAFNKEEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTDLAWEWWRTVIDDRNLPLVKNRNISIWGTTLYPKYSNKVDNPIE

(19) United States
(12) Patent Application Publication
(10) Pub. No.: US 2006/0204435 A1
(43) Pub. Date: Sep. 14, 2006

(54) Detection, imaging, and depletion of intracellular pathogens

(76) Inventor: Elizabeth S. Stuart, Amherst, MA (US)

Correspondence Address: FISH & RICHARDSON PC, P.O. BOX 1022, MINNEAPOLIS, MN, 55440-1022, US

(21) Appl. No.: 11/369,229

(22) Filed: Mar. 3, 2006

Related U.S. Application Data
(60) Provisional application No. 60/659,964, filed on Mar. 7, 2005.

(51) Int. Cl. A61K 51/00; G01N 33/554; G01N 33/569

(52) U.S. Cl. 424/1.49; 435/7.32

(57) ABSTRACT

Methods and compositions are disclosed for detecting and depleting cells infected with bacteria of the Chlamydiaceae family from a biological sample. Compositions include, for example, an immunoglobulin constant region polypeptide linked to an imaging moiety or a bactericide. Methods include, for example, contacting a biological sample that includes chlamydia infected cells, with a composition that includes an immunoglobulin constant region polypeptide linked to a detectable moiety, wherein the composition is selectively taken up by chlamydia infected cells and thereby detectably labels them. Methods of depleting chlamydia infected cells include for example, contacting a biological sample that includes chlamydia infected cells with a composition that includes an immunoglobulin constant region polypeptide linked to a bactericide, wherein the composition is selectively taken up by the infected cells and comes into contact with intracellular chlamydial bacteria and can thereby kill them or inhibit their replication.

### CLAIM OF PRIORITY

This application claims the benefit under 35 USC §119(e) of U.S. Provisional Patent Application Ser. No. 60/659,964, filed on Mar. 7, 2005, the entire contents of which are hereby incorporated by reference.

### TECHNICAL FIELD

This invention relates to methods and compositions for detecting, imaging, and depleting intracellular pathogens, particularly Chlamydia spp.

### BACKGROUND

Infections by members of the Chlamydiaceae family constitute a growing public health problem. Two key pathogens in humans are Chlamydia trachomatis, agent of trachoma and sexually transmitted disease, and Chlamydia pneumoniae, agent of community acquired pneumonia and a leading pathogen candidate for initiation or exacerbation of chronic diseases including atherosclerosis, cardiac artery disease, chronic obstructive pulmonary disease and neural pathologies such as multiple sclerosis and Alzheimer's disease.

The lack of methods to detect, image, and treat infectious as well as persistent chlamydia in subjects is a problem. Simpler identification assays are needed because these bacteria are “stealth” pathogens, frequently present, but not obviously in evidence. As a result, tests to detect these pathogens are often not performed. Further, certain tests are invasive, often requiring a biopsy followed by demonstration of the pathogen in tissue samples. In addition, standard treatments directed toward bacterial infections have proven to be relatively ineffective in treating chlamydial infections. Thus, new therapeutic approaches are needed that are specifically targeted against the peculiar life cycle of this pathogen.

### SUMMARY

The compositions and methods disclosed herein are based, in part, on the discovery that chlamydia infected cells specifically and selectively take up and accumulate immunoglobulins in intracellular, chlamydial inclusions. Without being bound by theory, the uptake of immunoglobulins into chlamydia infected cells appears to depend on the binding of the Fc region of immunoglobulins to an Fc receptor expressed on the cell surface of many types of cells including white blood cells. The selective uptake of immunoglobulins into chlamydial inclusions can be exploited for a number of purposes relating to the detection and imaging of chlamydial cells, for example in diagnostic assays for determining the presence of chlamydia in a subject. Methods for depletion of chlamydia infected cells from a population of cells are also disclosed.

Disclosed herein are molecular constructs that include a first portion including an amino acid sequence of an FcR-binding region of an immunoglobulin (e.g., an IgG, IgA, IgM, or IgE); this FcR-binding portion of the construct is referred to herein as an “FcR BP.” This portion of the construct serves to deliver the composition selectively into intracellular chlamydial inclusions within chlamydia infected host cells that express an Fc receptor. In some embodiments, the molecular construct includes a first FcR BP consisting of all or a fragment of an immunoglobulin heavy chain that binds to an FcR, and a second portion consisting of a cytotoxic moiety.

The FcR BP includes a first amino acid sequence that is at least 70% identical to all or part of an immunoglobulin heavy chain constant region (Hc), e.g., all or part of the Fc region, and retains the ability to bind to an Fc receptor.

In some embodiments, the first amino acid sequence is at least 75%, 80%, 85%, 88%, 90%, 92%, 95%, 96%, 97%, 98%, or 100% identical to all or part of an immunoglobulin heavy chain constant region, e.g., an IgQ, IgA, IgM, IgE, or IgG heavy chain constant region, e.g., all or part of the Fc region, or scFv with a Fc tail portion. Exemplary IgG immunoglobulin heavy chain constant region amino acid sequences are set forth in SEQ ID NOs:1-6. In general, constructs that include a first amino acid sequence from an IgG (e.g., at least 70% identical to all or part of an IgG Fc region therefrom) are able to bind to IgG Fc receptors (i.e., their “cognate” receptor), and constructs that include a first amino acid sequence from an IgA Hc are able to bind to IgA Fc receptors, and so on. In some embodiments, the first amino acid sequence includes the Fc region from an IgA, IgQ, IgD, IgE, or IgM immunoglobulin. In some embodiments, the first amino acid sequence does not include any of the antigen-binding sequence, i.e., does not includes any of the variable region.

Suitable second portions include, but are not limited to, therapeutic moieties, e.g., cytotoxic moieties, and detectable moieties, as described herein. In general the second moiety is linked to the first, FcR BP moiety in such a way that it does not interfere with binding of the targeting moiety to an Fc receptor; in some embodiments, a polypeptide or chemical linker is included between the two portions.

In some embodiments, the second portion includes a second amino acid sequence that is unrelated to an immunoglobulin heavy chain amino acid sequence (i.e., has less than 70% identity to an immunoglobulin amino acid sequence). In some embodiments, the second portion is not a protein, e.g., is an organic or inorganic molecule that is covalently bound (“conjugated”) to the targeting moiety.

In some embodiments, e.g., for therapeutic applications, the second portion can be a cytotoxin, e.g., a bactericide. In some embodiments, the cytotoxin is an intracellular cytotoxin, i.e., a cytotoxin that exerts its effect from inside the cell, e.g., by inducing apoptosis, impairing protein synthesis, causing free radical damage, impairing organellar function, e.g., mitochondrial or nuclear function. Exemplary cytotoxins include perfringolysin, a listeriolysin O (LLO) protein or biologically active fragment thereof, a fusion polypeptide of LLO or an LLO fragment and, e.g., a polypeptide toxin (e.g., Pseudomonas exotoxin, diphtheria toxin, cholera toxin, Shiga toxin 1, ricin, or a type I ribosomal inhibitor protein, from Mirabilis expansa (ME1)), calicheamicin, azithromycin, telithromycin, puromycin, doxycycline, linozelid, a proapoptotic protein e.g., granzyme B, granzyme M, caspase 3, or a Bcl2-homology-3 domain, or an enzyme that can break down a prodrug compound to yield a cytotoxin (e.g., herpes thymidine kinase, bacterial carboxypeptidase G2, alkaline phosphatase, or β-lactamase). Protein synthesis inhibitors (e.g., anisomycin or cycloheximide), or chemotherapeutic compounds (e.g., streptonigrin, bleomycin, tetrandrine, hypericin, maytansinoid 1, okadaic acid, or a tocotrienol) can also be used.

In some embodiments, e.g., where a detectable moiety is desirable, such as for use in detection and diagnostic methods, the second portion of the construct includes a second amino acid sequence that is a reporter protein, for example a fluorescent protein (e.g., enhanced green fluorescent protein, red fluorescent protein, cyan fluorescent protein, yellow fluorescent protein, and the like), a luciferase protein (e.g., firefly luciferase or renilla luciferase), and enzymes capable of yielding a detectable reaction product, e.g., alkaline phosphatases, horseradish peroxidase, β-galactosidase, or β-lactamase. In some embodiments, the construct includes a detectable moiety, e.g., fluorophores or detectable moieties suitable for in vivo imaging by X-ray imaging, magnetic resonance imaging, or positron emission tomography. Detection methods include, e.g., fluorescence microscopy, fluorescence activated, cell sorting, positron emission tomography, or magnetic resonance imaging.

In another aspect, the invention provides molecular constructs that include a first Fc receptor (FcR) binding portion consisting of all or a fragment of an immunoglobulin heavy chain that binds to an Fc receptor, linked to a nucleic acid molecule, e.g., a sequence encoding a therapeutic gene product (e.g., a peptide antibiotic or other therapeutic moiety described herein) or an antisense or RNAi construct that targets a chlamydia gene.

In some embodiments, the construct includes both a detectable moiety and a therapeutic moiety.

Compositions are also provided that include an antibody that selectively binds to chlamydia glycolipid exoantigen, and a bactericide linked to the antibody.

In an additional aspect, the invention includes pharmaceutical compositions including a molecular construct described herein and a pharmaceutically acceptable carrier. A therapeutically effective amount of such a composition can be administered to treat a chlamydia infection in a subject.

In a further aspect, methods are provided herein for detecting chlamydia in a cell. The methods include contacting a cell with a molecular construct as described herein that includes a detectable moiety, under conditions that allow the cell to internalize the construct. Subsequently, the presence of the detectable moiety in the cell is determined. The presence of the detectable moiety within an inclusion of the cell indicates that the cell is infected with chlamydia. These methods can be used to determine whether a subject is infected with chlamydia, e.g., when the cells are from a subject. The presence of the detectable moiety within the cells indicates that the subject is infected with chlamydia. In some embodiments, the cell is in a living subject, and determining the presence of the molecular construct inside the cell includes performing an in vivo assay to detect the detectable moiety, e.g., magnetic resonance imaging (MRI), positron emission tomography (PET), computed tomography (CT), or ultrasound.

In a further aspect, a method of tracking chlamydia infected cells in a subject is provided, in which the subject is administered a molecular construct as described herein that includes a detectable moiety. Subsequently, an in vivo assay, e.g., MRI, CT, PET or ultrasound, is conducted to detect the detectable moiety, thereby tracking the location of the infected cells.

Methods for separating chlamydia infected cells from uninfected cells are also provided herein. In these methods, a population of cells including chlamydia infected cells and uninfected cells is contacted with a molecular construct as described herein that includes a label, under conditions that allow the infected cells to internalize the construct. The infected cells are then separated from the uninfected cells based on the presence of the label in the infected cells. For example, where the label is a magnetic nanoparticle, a magnetic field is applied to the population of cells under conditions that allow separation of the population into infected cells that have internalized the polypeptide, and uninfected cells that have not internalized the polypeptide. Where the label includes a fluorescent moiety, separating the cells can be accomplished using, e.g., fluorescence-based cell sorting methods as are known in the art. In some embodiments, the population of cells is obtained from a subject. The methods can also include administering the uninfected cells to a subject in need thereof, thereby treating chlamydia in the subject.

In another aspect, the invention provides methods for treating a chlamydial infection in a subject, by administering to the subject a molecular construct as described herein that includes one or more cytotoxic moieties. In some embodiments, the methods include administering a standard treatment for chlamydia to the subject, e.g., an antibiotic, e.g., before, concurrently with, and/or after administration of the construct.

The methods described herein can be used to deplete chlamydia-infected cells from a population of cells, e.g., treating chlamydia in the cells to reduce the number of infected cells in the population, by contacting the cells with a construct described herein, e.g., that includes a therapeutic moiety or a label. Depletion of chlamydia infected cells from a population of cells can include specifically killing chlamydia cells (i.e., the pathogen itself), inhibiting replication of chlamydia, killing chlamydia infected cells, or selectively removing chlamydia infected cells from the population of interest. The methods can also be used to treat a subject infected with chlamydia, by administering to a subject a therapeutically effective amount of a construct described herein, e.g., a construct including a therapeutic moiety, e.g., a cytotoxin or nucleic acid.

In some embodiments, the methods described herein include using a mixture of constructs that includes constructs that will bind to more than one type of Fc, e.g., a mix of constructs including FcR binding fragments (or all) of two or more of an IgG, IgA, IgE, and/or IgM. In this way, multiple cell types can be targeted simultaneously. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-D are immunofluorescence photomicrographs of McCoy cells infected in culture with chlamydia C. trachomatis serovar K, and dual stained for chlamydia (red in original; 1B) and bovine immunoglobulin (green in original; 1A). A merged image, shown in 31, is overlaid with a differential interference contrast (DIC) image in 1D. Chlamydial inclusions can be clearly seen in two infected cells, marked with an arrow and “I,” in a field of a number of uninfected cells.

FIGS. 2A-D are confocal fluorescent images of chlamydia infected cells exposed to FITC-labeled goat IgG overnight. Anti Goat IgG (FITC labeled, green in original; 2A); anti-Chlamydia (TRITC labeled, red in original; 2C). An overlaid merged image is shown in 2D (the overlap was yellow in the original), and a DIC image is shown in 2B.

FIGS. 3A-D are images of white blood cell enriched samples dual immunostained to detect human IgG and Chlamydia. 3A, Anti-human IgG Fc specific mAb detected with a FITC conjugated anti-mouse secondary antibody. 3B, DIC image of the same field. 3C, An inclusion in an infected cell detected with a rabbit anti-Chlamydia antibody and a TRITC conjugated anti-rabbit secondary antibody. 3D, merge of images in A-C. White arrow (3A and 3C) shows no Chlamydia in a cell (an uninfected cell), black arrow (3B and 3D) shows no IgG inside the same cell. Asterisk indicates infected cell.

### DETAILED DESCRIPTION

The genus Chlamydia encompasses gram-negative obligate intracellular bacterial eukaryotic parasites that are associated with various chronic illnesses in humans and animals including infectious blindness, pneumonia, and sexually transmitted disease. A key aspect of the chlamydial infection cycle involves endocytosis of the infectious elementary body and rapid formation of a host derived membrane-bound parasitophorous vacuole, termed an inclusion. The inclusion creates an isolated niche separate from cell components, and allows Chlamydia to replicate without harm from host lysosomes and degradative enzymes, evading detection by the host immune system. Past research has shown that intermediate filament (IF) protein and β-catenin accumulate and co-localizes within the inclusions of infected cells.

To assess whether extracellularly derived proteins also can be accumulated in vitro, infected and uninfected cultured cells were co-immunostained for Chlamydia, and bovine immunoglobulin (Ig), an exogenous media component. Initial confocal microscopy indicated a co-association of bovine Ig and the inclusions of infected McCoy cells and J774A.1 macrophage cells, which both contain Fc receptors (FcRs). Microglial cells gave the same results, but infected Fc receptor negative Hec-1B cells showed no evidence of bovine Ig co-accumulation with the inclusions. As an indicator of uptake selectivity, inclusions were also screened for co-associated bovine serum albumin, a second media component and found no evidence it accumulates. When uninfected McCoy, J774A.1, and Hec-1B cells, were cultured using identical conditions, there was no evidence of accumulated bovine Ig or albumin within the cells. To test whether this finding was an artifact of in vitro culture, human donor blood smears were examined for evidence this phenomenon occurs in vivo. Smears from buffy coat preparations (WBC) were immunostained to detect intracellular Chlamydia and accumulations of human Ig. Dual stained smears clearly showed that most, but not all, Chlamydia-infected WBC also were positive for co-associated human Ig. Uninfected cells in these smears were all Ig negative, while smears of WBC demonstrated as Chlamydia-negative by PCR and immunostain, were uniformly Ig negative. Anti-human H&L and anti-human Fc-specific antibodies each labeled inclusion co-associated Ig so these sequences remain recognizably IgG. Thus, the results described herein indicate a specific, selective uptake that provides the ability to manipulate of host cell internalization and trafficking functions that in turn provides access to the inclusion compartment.

Compositions and methods are disclosed herein for detecting, tracking, quantifying, and/or depleting chlamydia infected cells ex vivo and in vivo. Compositions and methods are also disclosed for inhibiting chlamydial replication in chlamydia infected cells ex vivo and in vivo. Also disclosed are compositions and methods for separating a first population of cells containing a fraction of chlamydia infected cells into a second population of cells substantially free of chlamydia and a third population enriched for chlamydia infected cells. Finally, therapeutic methods are disclosed for treating chlamydia infection in a subject.

I. Molecular Constructs

As demonstrated herein, molecular constructs that include an FcR-binding part of a constant region of immunoglobulin heavy chain (C_{H}) bind to cell surface Fc receptors and are taken up selectively by chlamydia infected cells and localized to chlamydial inclusions. Constructs that have the amino acid sequence of all or part of a FcR-binding portion of C_{H} domain or a closely related sequence thereto are therefore useful for delivering compositions to chlamydia infected cells that express an FcR, e.g., monomericFcR (mFcR) or polymeric FcR (pFcR), including the ‘neonatal’ FcR (FcRn). The disclosed constructs include all or part of a C_{H} domain, with an amino acid sequence that is at least 70% identical to all or a part of the amino acid sequence of an immunoglobulin heavy chain constant region sequence that binds to extracellular FcR, e.g., mFcR or pFcR, including FcRn.

The constructs can include all or part of an immunoglobulin heavy chain constant region that binds FcR can be used to target the construct to chlamydia infected cells (e.g., whole antibodies or fragments thereof, including all or part of the HC, all or part of the Fc, an scFv with a Fc tail portion, or any other configuration or fragment that retains FcR-binding ability). Constructs can be dimerized by means of disulfide bonds cross-linking.

In some embodiments, the FcBP portion of the constructs does not include antigen-specific variable regions (e.g., lacks the Fab). In some embodiments, the construct includes an entire intact antibody.

In some embodiments, the construct includes all or part of an antibody directed against the chlamydial glycolipid exoantigen as disclosed in pending U.S. patent application Ser. No. 09/827,490 to Stuart et al.

Purified polypeptides include polypeptides that are generated in vitro (e.g., by in vitro translation or by use of an automated polypeptide synthesizer) and polypeptides that are initially expressed in a cell (e.g., a prokaryotic cell, a eukaryotic cell, an insect cell, a yeast cell, a mammalian cell, a plant cell) and subsequently purified. Implementations of cells expressing a molecular construct described herein include, for example, cells transduced with an expression vector encoding the construct. In some implementations, the cell expresses a fusion protein (e.g., FcR BP-GST fusion) that includes a protease cleavage site to allow cleavage and separation of the fusion protein into separate polypeptides. In some embodiments, the constructs described herein include an amino acid sequence that facilitates purification of the polypeptide (e.g., a multiple histidine tag, or a FLAG tag). Methods for isolating proteins from cells or polypeptides that are expressed by cells, include affinity purification, size exclusion chromatography, high performance liquid chromatography, and other chromatographic purification methods. The polypeptides can be post-translationally modified, e.g., glycosylated.

To determine the percent identity of two amino acid sequences, the sequences are aligned for optimal comparison purposes (e.g., gaps can be introduced in the sequence of a first amino acid sequence for optimal alignment with a second amino sequence). The amino acid residues at corresponding amino acid positions are then compared. When a position in the first sequence is occupied by the same amino acid residue as the corresponding position in the second sequence, then the molecules are identical at that position. The percent identity between the two sequences is a function of the number of identical positions shared by the sequences (i.e., % identity=number of identical positions/total number of positions×100).

As used herein, “percent homology” of two amino acid sequences is determined using the algorithm of Karlin and Altschul (1990) Proc. Nat'l Acad. Sci. USA 87:2264-2268, modified as in Karlin and Altschul (1993) Proc. Nat'l Acad. Sci. USA 90:5873-5877. Such an algorithm is incorporated into the NBLAST and XBLAST programs of Altschul, et al., (1990); J. Mol. Biol. 215:403-410. BLAST protein searches are performed with the XBLAST program, score=50, wordlength=3 to obtain amino acid sequences homologous to a reference polypeptide. To obtain gapped alignments for comparison purposes, Gapped BLAST is utilized as described in Altschul et al., (1997) Nucleic Acids Res. 25:3389-3402. When utilizing BLAST and Gapped BLAST programs, the default parameters of the respective programs (e.g., XBLAST) are used. See the World Wide Web at address ncbi.nlm.nih.gov.

The molecular constructs described herein can include all or part of the immunoglobulin heavy chain constant regions, that bind to their cognate FcR. Most mammalian immunoglobulin (Ig) heavy chains have three to four Ig-like domains of conserved sequence termed C_{H1} (Constant heavy 1) to C_{H4} (Constant heavy 4); these domains include the regions important for binding to Fc Receptors on the surface of a cell. A “hinge” region separates the C_{H1} and C_{H2} domains. The portion of an immunoglobulin comprising the hinge region plus the domains C_{H2} and C_{H3} (and C_{H4}, in IgM) is called fragment crystallizable (Fc); constructs including only the Fc are suitable for use herein. There are several different human and other mammalian (e.g., murine) Ig molecules, including IgA, IgG, IgE, IgD, and IgM. Several immunotherapeutic agents for human therapy include the human IgG1 Fc portion. All or a portion of the HC, e.g., all or a portion of the Fc region, is used to prepare the FcR binding moiety of the molecular constructs described herein. The FcR binding moiety must retain a sufficient amount of the HC to bind to an FcR; methods for making, testing, and selecting suitable fragments are known in the art. For example, a fragment of an Fc can be evaluated for its ability to bind to Fc in a standard binding assay.

Mutated Fc regions can also be used, e.g., Fc regions that bind to with the same or higher affinity to FcR. For example, mutated Fc are described in Vaccaro et al., Nat. Biotechnol. 23(10)1283088 (2005).

In some embodiments, the subject's own immunoglobulins can be used to generate the molecular constructs described herein, e.g., for delivery of cytotoxic or imaging moieties. A biological sample containing an immunoglobulin is first obtained from the subject using appropriate sterile technique (e.g., from a sample of serum). The subject's immunoglobulins can then be purified by any number of standard techniques, e.g., affinity chromatography using protein A (see, e.g., Harlow et al., Antibodies: A Laboratory Manual). The purified immunoglobulins are then conjugated to any of the cytotoxic or detectable moieties described above for molecular constructs. The detectably labeled immunoglobulins are then introduced back into the subject, e.g., by injection and subsequently detected and imaged at various time points thereafter. In other implementations, a subject with a chlamydial infection is administered a construct composition that includes a detectable moiety, such as one of those described above (e.g., ⁴⁵Ca). Detection and/or imaging can be performed at least one hour after administration to and repeated at subsequent time points (e.g., 12 hours, 24 hours, 36 hours, 42 hours, etc). Specific times will vary depending on the moiety (e.g., the sensitivity of the detection method). Routes of administration and formulations of construct compositions are described in more detail below.

In still other implementations immunoglobulins are conjugated to an imaging moiety that can be visualized by PET or MRI, for example a gadolinium complex such as Gd-DTPA. PET and MRI can also permit anatomical localization and tracking of the cells over time. MRI is also suitable for analysis of chlamydia infections that have been associated with atherosclerosis, Alzheimer's disease, multiple sclerosis, and asthma. See for example, Mitusch et al., (2005) Arterioscler. Thromb Vasc Biol., 25(2):386-391, Balin et al., (1998) Med Microbiol Immunol (Berlin), 187(1):23-42; Contini et al, (2004) Mult. Scler., 10(4):360-369.

The molecular constructs described herein also include a second portion that is not related to an immunoglobulin heavy chain or light chain sequence, that includes a detectable moiety or a therapeutic moiety. The second portion can be a peptide or polypeptide, and thus can be produced as part of a fusion protein with the FcR BP. Methods for producing such conjugates are known in the art, see, e.g., Dumont et al., J. Aerosol Med. 18(3):294-303 (2005), and Low et al., Hum Repro. 20(7):1805-1813 (2005). Alternatively, the second portion can conjugated to the FcR BP, e.g., chemically conjugated. Methods for preparing such conjugated molecular constructs are also known in the art.

This second region can be fused or attached to the C-terminus or the N-terminus of the Fc targeting moiety, with or without an intervening linker (e.g., a poly-lysine or poly-alanine linker or DOTA linker), so as not to interfere with binding of the molecular construct to an FcR. All or part of a third region, e.g., a peptide, may also be present. Thus, a construct can include an Fc targeting moiety fused to a second polypeptide, e.g., glutathione-S-transferase (GST), a reporter polypeptide (e.g., a green fluorescent protein variant, a luciferase, an alkaline phosphatase, or β-galactosidase), a short amino acid sequence tag, or a polypeptide toxin. Methods for generating fusion polypeptides by recombinant DNA methodologies are well known and described, for example in Sambrook et al., Molecular Cloning: A Laboratory Manual, 2nd, ed., Cold Spring Harbor Laboratory, Cold Spring Harbor Laboratory Press, Cold Spring Harbor, N.Y., (1989), incorporated herein by reference.

In one example, a molecular construct as described herein can be made by cloning into an expression vector such as pcDNA3 (Invitrogen) a nucleic acid sequence encoding a second moiety in-frame with a sequence encoding all or part of an Fc portion of an Ig (e.g., the Fc portion of an Ig such as an IgG, IgA, IgE, or IgM).

For example, imaging moieties and cytotoxic compounds can be linked to a construct by non-covalent means, by attaching both the construct and a moiety (e.g., a detectable moiety) to an electrostatically charged carrier molecule (e.g., poly-lysine), as described in U.S. patent application Ser. No. 10/793138 to Waugh and Dake.

II. Labeled Molecular Constructs

In some embodiments, e.g., for detection, imaging, and separation implementations, a molecular construct as described herein can include a second portion that includes a label.

The disclosed polypeptides can also be labeled with various moieties that are selected based on the particular application (e.g., ex vivo or in vivo), the condition being diagnosed or imaged, the spatial and temporal sensitivity of detection required, the imaging resolution required, the route of administration, and the like.

In some embodiments, the label includes an enzyme capable of yielding a detectable reaction product, e.g., alkaline phosphatases, horseradish peroxidase, β-galactosidase, or β-lactamase.

In some embodiments, the label includes a fluorophore, e.g., a polypeptide that forms a fluorescent protein, or an enzyme capable of yielding a detectable reaction product. A number of such polypeptides are known in the art. Examples include, but are not limited to, fluorescent proteins (e.g., enhanced green fluorescent protein, red fluorescent protein, cyan fluorescent protein, yellow fluorescent protein, or variants thereof), or luciferase (e.g., firefly luciferase and renilla luciferase).

In some embodiments, the molecular constructs can be labeled with a fluorophore that emits light of a particular color, e.g., a color that contrasts with other fluorophores. Techniques for labeling polypeptides (e.g., antibodies), are described, for example, in Harlow et al., Antibodies: A Laboratory Manual, Cold Spring Harbor Laboratory, Cold Spring Harbor, N.Y., 353-355 (1988) and see also, The Handbook—A Guide to Fluorescent Probes and Labeling Technologies, Molecular Probes, Inc., Eugene, Oreg., (2004), incorporated herein by reference. For example, polypeptides can be labeled with one or more of the following fluorophores: 7-amino-4-methylcoumarin-3 -acetic acid (AMCA), Texas Red™ (Molecular Probes, Inc., Eugene, Oreg.), 5-(and-6)-carboxy-X-rhodamine, lissamine rhodamine B, 5-(and-6)-carboxyfluorescein, fluorescein-5-isothiocyanate (FITC), 7-diethylaminocoumarin-3carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5(and-6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, phycoerythrin (B—, R—, or cyanine-), allophycocyanin, Oregon Green™, Cascade™ blue acetylazide, Alexa Fluor Dyes™ (Molecular Probes, Inc., Eugene, Oreg.), cyanine dyes, e.g. Cy3™, Cy5™ and Cy7™ dyes (Amersham Biosciences, UK, LTD), and near infrared cyanine fluorochromes as described in Lin et al., 2002, Bioconjugate Chem., 13:605-610.

Alternatively or in addition, polypeptides can be labeled with semiconductor nanocrystals, also known as quantum dots. Water soluble nanocrystals are composed of different sizes of cadmium-selenium/cadmiumsulfur core-shell nanocrystals enclosed in a silica shell or cadmium-selenium/zincsulfur nanocrystals solubilized in mercaptoacetic acid. Such water soluble nanocrystals have a narrow, tunable, symmetric emission spectrum and are photometrically stable. See, Bruchez Jr. et al., Science 281:2013-2016 (1998); and Chan et al., Science 281:2016-2018 (1998), both of which are incorporated herein by reference in their entirety.

Examples of moieties particularly suitable to implementations related to in vivo detection and/or imaging of chlamydia infected cells, include, inter alia, radiopaque contrast agents, paramagnetic contrast agents, superparamagnetic contrast agents, and computerized tomography (CT) contrast agents.

Examples of radiopaque contrast agents (for X-ray imaging) include inorganic and organic iodine compounds (e.g., diatrizoate), radiopaque metals and their salts (e.g., silver, gold, platinum and the like) and other radiopaque compounds (e.g., calcium salts, barium salts such as barium sulfate, tantalum and tantalum oxide). Suitable short-lived radioisotopes include, e.g., ⁴⁵Ca, ⁶⁴Cu, ¹²³I, ⁷⁶Br. Radiolabels suitable for positron emission tomography (PET), included, e.g., ¹¹C and ¹⁸F.

Suitable paramagnetic contrast agents (for magnetic resonance imaging) include gadolinium diethylene triaminepentaacetic acid (Gd-DTPA) and its derivatives, and other gadolinium, manganese, iron, dysprosium, copper, europium, erbium, chromium, nickel and cobalt complexes, including complexes with 1,4,7,10-tetraazacyclododecane-N,N′,N″,N′″-tetraacetic acid (DOTA); ethylenediaminetetraacetic acid (EDTA), 1,4,7,10-tetraazacyclododecane-N,N′,N″-triacetic acid (DO3A), 1,4,7-triazacyclononane-N,N′,N″-triacetic acid (NOTA), 1,4,8,11-tetraazacyclotetradecane-N,N′,N″,N′″-tetraacetic acid (TETA), hydroxybenzylethylene-diamine diacetic acid (HBED) and the like.

Suitable superparamagnetic contrast agents (for magnetic resonance imaging) include magnetites, superparamagnetic iron oxides, or monocrystalline iron oxides, particularly complexed forms of each of these agents that can be attached to a negatively charged backbone.

Still other suitable imaging agents are the CT contrast agents including iodinated and noniodinated and ionic and nonionic CT contrast agents, as well as contrast agents such as spin-labels or other diagnostically effective agents. Methods for coupling these agents to polypeptides can be found, for example, in U.S. Pat. No. 5,900,228 to Meade et al., incorporated herein by reference.

For use in separation methods, the constructs can include any label that allows separation, e.g., a fluorophore that can be used in a fluorescence-based cell sorting or counting method, or a collectible moiety, e.g., a magnetic nanoparticle.

III. Molecular Constructs Including Cytotoxic Moieties

In other implementations, e.g., for therapeutic applications, a molecular construct can include a cytotoxic moiety. In some embodiments, the cytotoxic moiety is a cytotoxic polypeptide.

In one example, a cytotoxic polypeptide includes the amino acid sequence of a listeriolysin O (LLO) polypeptide or a biologically active fragment thereof. The usefulness of listeriolysin as a phagosomal permeabilizer has been described in U.S. Pat. No. 5,643,599 to Lee et al., which is herein incorporated by reference. LLO or perfringolysin, and fragments thereof, can be useful for permeabilizing chlamydial inclusions to release their contents into the host cell cytoplasm. In some implementations, LLO or perfringolysin can act as a host cell toxin, lysing the chlamydia infected host cell and thereby preventing replication of chlamydia in that host cell.

In some embodiments, the cytotoxic moiety is an LLO polypeptide that lacks the proline-glutamate-serine/threonine (PEST) protein degradation motif present in the wild type LLO sequence. The absence of the PEST sequence in LLO, which normally reduces the half life of the protein, allows the LLO to kill cells by perforating cell and organelle membranes, thereby leading to cell death by lysis (see, e.g., Decatur et al., (2000) Science, 290:992-995). The function of LLO in perforating cellular membranes can also be exploited to allow macromolecules to exit intracellular chlamydial inclusions and thereby enter the host cell cytoplasm. For example, LLO can be fused to a toxic polypeptide, as describe above, and thereby promote export of the toxic LLO fusion polypeptide from a chlamydial inclusion into the host cell cytoplasm and/or other host cell compartments. In one implementation, the LLO amino acid sequence contains a G486D substitution, described in Decatur et al., (2000), supra, that reduces the excessive lytic activity of LLO, thereby permitting the escape of the fusion polypeptide from the chlamydial inclusion, while reducing or eliminating host cell lysis by LLO. This is useful, e.g., where it is desirable to allow a polypeptide fused to LLO to function in the host cell cytoplasm, e.g., proapoptotic proteins (e.g., caspase 3), prodrug enzymes (e.g., herpes thymidine kinase), or polypeptide toxins (e.g., Shiga toxin 1).

Suitable FcR BP-LLO fusion polypeptides include polypeptides that include an LLO amino acid sequence that is at least 90%, 92%, 94%, 96%, 98%, or 100% identical to that of SEQ ID NO:7 or SEQ ID NO:8. In further implementations, a fragment of a listeriolysin O polypeptide with an amino acid substitution is useful in permitting the Fc target polypeptide fusion polypeptide to exit from the chlamydial inclusion without causing cell death by excessive lytic activity.

In some embodiments, the molecular construct can include an enzyme that can convert a non-toxic prodrug into an active cytotoxic compound, for example herpes thymidine kinase, which can act on the prodrug ganciclovir. In yet other implementations, the fused polypeptide is a toxin (e.g., Pseudomonas exotoxin, diphtheria toxin, or cholera toxin) effective for killing a eukaryotic cell when inside the cell, or a proapoptotic protein (e.g., granzyme B, granzyme M, caspase 3, or a Bcl2-homology-3 domain ). In one implementation, a polypeptide toxin can be a ribosomal inhibitor protein (RIP) (e.g., Shiga toxin 1, ricin, or a type I RIP (ME1), from Mirabilis expansa).

In some implementations, the molecular construct includes a conjugates of an FcR BP and a non-polypeptide cytotoxic moiety. Suitable non-polypeptide cytotoxic moieties include bacteriocidal/bacteriostatic compounds, for example antibiotics (e.g., nitroimidazoles, nitrofurans, isoniazid, aconizaide; pyrazinamidy, calicheamicin, puromycin, doxycycline, linozelid, macrolides sudran, azithromycin telithromycin, orketolides such as cethromycin or telithramycin) or protein synthesis inhibitors (e.g., anisomycin or cycloheximide), see e.g., U.S. Pat. Pub. No. 2005/042690. In other implementations, the cytotoxic moiety can a compound effective for killing a eukaryotic cell (e.g., a chemotherapeutic compound). Suitable chemotherapeutic compounds include, e.g., calicheamicin (also an antibiotic), streptonigrin, bleomycin, tetrandrine, hypericin, maytansinoid 1, okadaic acid, or a tocotrienol. Methods for conjugating cytotoxic compounds to polypeptides are well known in the art. See, for example, U.S. Pat. No. 5,087,616 to Myers et al., DiJoseph et al., Cancer Immunol. Immunother. 54(1):11-24 (2005); and Komissarenko et al., Int. J. Immunopharmacol. 16(12):1053-8(1994). In some implementations, a link between the FCR BP portion of the molecular construct and the cytotoxic moiety can be a hydrolysable (e.g., an ester bond) or reducible (e.g., disulfide) bond/linkage (see e.g., U.S. patent application Ser. No., 10/835151), which may permit release of the cytotoxic moiety from the construct, once the composition is inside an intracellular compartment (e.g., a chlamydial inclusion).

In some embodiments, the molecular constructs include both a therapeutic moiety and a detectable moiety.

IV. Molecular Constructs Including Nanoparticles

In some implementations, the molecular constructs described herein can include or be conjugated to a nanoparticle, which can act as a suitable carrier for any of the imaging or cytotoxic moieties described supra. Methods for generating biologically compatible nanoparticles and methods for conjugating nanoparticles to polypeptides are known in the art (see, for example, U.S. Pat. No. 5,565,215 to Gref et al.). The nanoparticles can be magnetic nanoparticles which are particularly useful as labels in cell separation applications, e.g., as described in U.S. Pat. No. 6,797,514 to Berenson et al., In some implementations, a construct is first biotinylated and subsequently bound to an avidin modified nanoparticle bearing an imaging moiety (e.g., a fluorophore) or a cytotoxic moiety (e.g., an antibiotic). See, for example, Balthasar et al., Biomaterials 26:2723-2732 (2005), Huhtinen et al., J. Immunol. Methods, 294(1-2):111-122, (2004), and Zhao et al., PNAS, 101(42):15027-15032, (2004), which are incorporated herein by reference.

V. Molecular Constructs Including Conjugates of FcR BPs to Nucleic Acids

A current limitation of methods that target chlamydia is the absence of a means to genetically manipulate the pathogen. As an EB, chlamydia is essentially impervious to the introduction of plasmid DNA and previously there was no way to target such DNA into the inclusion so as to increase the probability of getting exogenous DNA incorporated into the replicating chlamydial ‘chromosomes’ during the replicative cycle.

Described herein are molecular constructs that include FcR BP that are conjugated to a nucleic acid, e.g., DNA or RNA, and the resulting nucleic acid-protein fusion molecule can be used, e.g., to deliver the nucleic acid to any cell that expresses FcR on its surface and internalizes the FcR, e.g., chlamydia infected cells; for example, these molecules can be used to deliver nucleic acids to chlamydial inclusions. For example, antisense DNA or RNA, plasmid DNA, siRNA or dsRNA, or DNA encoding an antisense or other inhibitory nucleic acid molecule such as an siRNA, can be conjugated to the FcR BP. Internalized IgG undergoes degradation once taken into the inclusion, thereby freeing the nucleic acid once it is within the inclusion.

In some embodiments, the nucleic acid is or includes DNA that encodes a protein cytotoxin or reporter protein as described herein.

In some embodiments, the nucleic acid is used to disrupt a chlamydia gene. For example, the nucleic acid can be constructed such that it will be integrated into the genome of the chlamydia; in this case, the nucleic acid can be designed to insert one or more stop codons in a vital gene or genes, e.g., a heat shock protein or proteins necessary for replication. In some embodiments, the nucleic acid targets an origin of replication of the chlamydia genome, to disrupt replication. In some embodiments, the nucleic acid targets a gene that codes for the chlamydial protease-like activity factor (CPAF). CPAF is a degradative enzyme that turns off the formation of MHC I and II in the host cell, by degrading the relevant host cell transcription factor. Targeting the major outer membrane protein (MOMP) would be expect to prevent the formation of new infectious EBs. This would be true for certain other Chlamydia gene products as well, e.g., a porin gene, or any of the type III secretory apparatus genes.

Nucleic acids that are inserted into the chlamydia genome can also be used for tracking, e.g., to track infected cells or to follow an inserted sequence through multiple generations of chlamydia.

Methods for conjugating nucleic acids to proteins are known in the art, see, e.g., Doi and Yanagawa, FEBS Lett., 457(2):227-30 (1999); Yonezawa et al., Nucleic Acids Research, 31(19):e118 (2003); Bolesta et al., Virology, 332(2):467-79 (2005); Burbulis et al., Nat. Methods, 2(1):31-7 (2005); Sloots and Wels, FEBS J., 272(16):4221-36 (2005); and Uherek et al., J. Biol. Chem., 273(15):88357-41 (1998). See also Sebestik et al., Biopolymers. Feb. 23, 2006; [Epub ahead of print], which describes methods for generating dsDNA binding peptides.

Alternatively, the methods described herein can include targeting DNA, e.g., plasmid DNA, to chlamydia infected cells by administering a complex that includes the DNA and an anti-DNA antibody. In such a case, the DNA/antibody complex is internalized via the Fc region of the antibody. Methods for generating such antibodies are known in the art; see, e.g., Komissarov et al., J. Biol. Chem. 271 (21):12241-12246 (1996); Schuermann et al., J Mol Biol. 347(5):965-78 (2005); Paz et al., Mol. Cancer Ther. 4(11): 1801-9 (2005); and Vaz de Andrade et al., Biochim Biophys Acta. 1726(3):293-301 (2005).

VI. Methods for Detecting, Imaging, and Selecting Chlamydia Infected Cells Methods for detecting and imaging chlamydia infected cells can enable medical practitioners to diagnose whether a subject (a) is or is not currently infected with bacteria of the chlamydiaceae family; and (b) if the subject is infected, the subject's infection status. In determining a subject's infection status, a determination can be made as to how many bacteria a subject carries (i.e., a subject's “chlamydial load”). If a subject carries a relatively high chlamydial load, the subject may be a symptomatic carrier of the bacteria (i.e., the subject may exhibit outward signs of the disease). If the subject carries a relatively low chlamydial load, the subject may have recently been infected or may be an asymptomatic carrier of chlamydia. The methods are useful for diagnosing subjects as being carriers of chlamydia, i.e., as persistently carrying a chlamydial load high enough to allow transmission to others but low enough that the subject does not display disease symptoms. Where a subject has undergone, is undergoing, or will undergo a therapeutic treatment to reduce/eliminate chlamydia, the methods are particularly useful for monitoring the effectiveness of the therapeutic treatment. The methods can also be useful for tracking the distribution of chlamydia infected cells in a subject and may also be useful to generate a diagnostic correlate to other disease states including atherosclerosis, multiple sclerosis, and Alzheimer's dementia, since there is evidence that cells affected in these pathologies are frequently infected with chlamydia.

In the disclosed methods, any polypeptide that binds to FcR domain can be used to target a composition to chlamydia infected cells (e.g., antibodies or fragments thereof, the disclosed molecular constructs or fragments of the disclosed constructs that can bind to an Fc receptor). Methods for generating immunoconjugates using antibodies or fragments thereof are well known in the art and are similar to those described for generating compositions including the molecular constructs described herein. In some embodiments, the methods include using a mixture of constructs that includes constructs that will bind to more than one type of Fc, e.g., a mix of constructs including FcR binding fragments (or all) of two or more of an IgG, IgA, IgE, and/or IgM. In this way, multiple cell types can be targeted.

Detection and/or imaging of chlamydia infected cells can be accomplished by assaying cells for uptake of a detectably labeled molecular construct or an antibody. Depending on the particular application, the disclosed methods can be used to detect infected cells ex vivo, in vivo, or both. Species of chlamydia that can be detected or imaged with the disclosed methods include, but are not limited to, Chlamydia trachomatis, Chlamydia suis, Chlamydia muridarum, Chlamydophilia psittaci, Chlamydophilia pneumoniae Chlamydophilia caviae, Chlamydophilia pecorum, Chlamydophilia abortus, and/or Chlamydophilia felis. The disclosed methods are particularly useful to detect chlamydia infected cells that express an Fc receptor including primary cells (e.g., B lymphocytes, dendritic cells, macrophages, monocytes, eosinophils, natural killer cells, neutrophils, mast cells, langherhans cells, platelets, endothelial cells, mesangial cells, or sperm cells) and cells derived from a suitable cell line (e.g., McCoy cells, Baby Hamster Kidney cells, or HeLa cells).

Methods for detecting and imaging chlamydia infected cells can involve exposing a biological sample to one of the detectably labeled constructs described herein, and determining the presence of the construct in cells by any of a number of detection and/or imaging assays appropriate to the detectable moiety. In some implementations, cells can be cultured in vitro, and infected cells can be identified by contacting and incubating the cultured cells (e.g., for at least one hour) with one of the disclosed molecular constructs that includes a detectable moiety. Unbound excess extracellular molecular constructs can be removed by repeated washing (e.g., 3 times) with an appropriate physiological buffer.

Examples of detection methods include fluorescence microscopy, confocal microscopy, and flow cytometry, or any variation thereof. Particularly suitable implementations for detecting Chlamydia infected cells ex vivo include fluorescence based assays, including, for example, fluorescence microscopy and/or fluorescence activated cell sorting (FACS). Methods for performing fluorescence microscopy to detect chlamydia-infected cells, and performing flow cytometry, are well known in the art and are described, for example, in Norkin et al., Exp. Cell. Res. 266(2):229-38 (2001); Handbook of Flow Cytometry Methods. J. Paul Robinson (Editor) Wiley (1993); and McLean et al., Marcel Dekker, Inc, New York, (1990); Poccia et al., Emerging Infectious Diseases, 9(11):03-0349 (2003); and Mandy et al., Guidelines for the Performing Single-Platform Absolute CD4⁺ T-Cell Determinations with CD45 Gating for Persons Infected with Human Immunodeficiency Virus; January 2003/52 (RR02);1-13. Morbidity& Mortality Report. Methods for the detection of Chlamydia in the peripheral blood cells of normal donors using in vitro culture, immunofluorescence microscopy and flow cytometry techniques are described in Cirino et al., BMC Infect Dis. 6(l):23 (2006) (Epub ahead of print as doi:10.1186/1471-2334-6-23).

Typically, in flow cytometry, cells (or cellular fragments) labeled with an internalized fluorescent moiety are passed through a slender flow cell along with a sheath fluid so that the cells flow in single file. The individual cells in the flow are irradiated one at a time with a light beam (such as a laser beam) by means of hydrodynamic focusing, and the intensity of scattered light or fluorescent light from the cells, e.g., light information indicative of the cells, is measured instantaneously to analyze the cells. Flow cytometry of this kind is advantageous in that a large number of cells can be analyzed at high speed and with great accuracy.

Flow cytometers are well known in the art and are commercially available from, e.g., Beckman Coulter and Becton, Dickinson and Company. Typical flow cytometers include a light source, collection optics, electronics and a computer to translate signals to data. In many cytometers, the light source of choice is a laser which emits coherent light at a specified wavelength. Scattered and emitted fluorescent light is collected by two lenses (one set in front of the light source and one set at right angles) and by a series of optics, beam splitters and filters, specific bands of fluorescence can be measured.

One known example of a cell analyzing apparatus using flow cytometry comprises a flow cell for forming a slender stream of liquid, a light source (such as a laser) for irradiating the cells which flow through the interior of the flow cell with a light beam, a photodetector for detecting cell light information from the cells irradiated with the light beam and converting the light information into an electric signal, a signal processing circuit for amplifying, integrating and removing noise from the signal produced by the photodetector, and a computer for processing a signal, which represents the cell light information, outputted by the signal processing circuit.

Skilled practitioners will appreciate that many variations and/or additions to basic flow cytometry systems can be made, e.g., providing practitioners with additional and/or different analyzing capabilities. Further, skilled practitioners will appreciate that flow cytometry can be performed in an automated manner and that a flow cytometer can be provided as part of a larger, automated system, e.g., a high-throughput system. The methods of the present invention contemplate the use of such apparatus and systems. Also included within the present invention is the use of any apparatus not known as a flow cytometer, but which performs essentially the same function as a flow cytometer, as described above.

In some implementations, FACS is used to separate a population of cells containing chlamydia infected cells into separate subpopulations of cells that are enriched for infected or uninfected cells. FACS of the enriched subpopulation of uninfected cells can be repeated multiple times until an acceptably low fraction of infected cells are present in the uninfected cell subpopulation (e.g., testing for the presence of chlamydia infected cells by quantitative polymerase chain reaction with Chlamydiales-specific primers for 16S ribosomal RNA). Cell populations actively selected so as to contain acceptably low levels of chlamydia contamination are useful, for example, in animal husbandry or therapeutic applications in which donor cells that are infected with chlamydia might otherwise be transferred to a recipient (e.g., sperm cells, blood cells or stem cells). In one implementation, chlamydia infected cells can be selected out of a population of cells by first contacting the cells with a molecular construct that includes a magnetic nanoparticle. Chlamydia infected cells that take up the construct-magnetic nanoparticle composition can then be easily selected by applying a magnetic field to the mixed population of infected and uninfected cells. Details of the use of magnetic nanoparticles for cell separation applications are described, e.g., in U.S. Pat. No. 6,797,514 to Berenson et al.

In other implementations, chlamydia infected cells can be detected in a subject in vivo. The subject can be an experimental animal (e.g., a rodent) and in other implementations it can be a human subject. Detection methods can involve obtaining a biological sample comprising an immunoglobulin from a subject, purifying immunoglobulin therefrom and directly labeling the subject's own immunoglobulin. A solution with the labeled immunoglobulin can then be introduced into the subject (e.g., the subject's serum) and the distribution of the labeled immunoglobulin can then be assayed in vivo.

Detectably labeled compositions can be administered to a living subject and subsequently the distribution of the composition in vivo can be determined, preferably using a non-invasive imaging method such as magnetic resonance imaging (MRI), positron emission tomography (PET), or computed tomography (CT), or ultrasound. Immunoglobulins that are conjugated to an imaging moiety that can be visualized by PET or MRI, for example a gadolinium complex such as Gd-DTPA. PET and MRI, can also permit anatomical localization and tracking of the cells over time. MRI is also suitable for analysis of chlamydia infections that have been associated with atherosclerosis, Alzheimer's disease, multiple sclerosis, and asthma. See for example, Mitusch et al., (2005) Arterioscler. Thromb Vasc Biol., 25(2):386-391, Balin et al., (1998) Med Microbiol Immunol (Berlin), 187(1):23-42; Contini et al, (2004) Mult. Scler., 10(4):360-369.

In other implementations, infected cells can be tracked in vivo by imaging a molecular construct that is conjugated to an imaging moiety that includes a fluorophore that fluoresces in the infrared spectrum (e.g., a near infrared cyanine fluorochrome). Methods for near infrared fluorescence imaging of labeled cells in subject (e.g. a rat) are described in U.S. Pat. No. 6,592,847 to Weissleder, et al. and in Moon et al., (2003) Bioconjugate Chem., 14:539-545. Imaging in the near infrared spectrum is particularly useful for detecting chlamydia infected cells that are located superficially in the subject, for example in cells flowing through blood vessels that run throughout the skin.

VII. Methods for Treating a Chlamydia Infection

The present methods are useful for specifically targeting and depleting the number of chlamydia infected cells in a population of cells (e.g., in culture, or in a subject, e.g., a human or animal subject that is infected with chlamydia, e.g., a subject selected on the basis that they are infected with chlamydia). As disclosed above, any polypeptide that includes an FcR BP can be used to target a composition to chlamydia infected cells (e.g., antibodies or fragments thereof, the disclosed constructs, or fragments of the disclosed constructs that can bind to an Fc receptor). A population of cells that includes chlamydia infected cells can be contacted with a molecular construct described herein, e.g., a construct that includes a therapeutic moiety, e.g., a toxin lethal to prokaryotic and/or eukaryotic cells when present intracellularly, e.g., as described herein.

In some embodiments, cells are contacted with a molecular construct including a therapeutic moiety as described herein, such as a construct including a bacteriocidal or bacteriostatic compound, e.g., an antibiotic (e.g., calicheamicin, azithromycin, telithromycin, or doxycycline) or a cytotoxic compound such as a chemotherapeutic compound that can kill the host cell and prevent further chlamydial replication. Examples of such cytotoxic compounds include e.g., chemotherapeutic compounds (e.g., calicheamicin, streptonigrin, bleomycin, tetrandrine, hypericin, maytansinoid 1, okadaic acid, or a tocotrienol).

In some embodiments, treatment of chlamydia infection includes selecting out chlamydia infected cells from a population of cells that contains uninfected and chlamydia infected cells. For example FACS or magnetic nanoparticle separation can be used to select out chlamydia infected cells and obtain a population of cells enriched for uninfected cells.

When chlamydia infected cells are present in a subject (e.g., an experimental animal, a human subject), the disclosed methods can be used to reduce the number of or eliminate chlamydia infected cells in the subject. For example, the molecular constructs described herein can be directly administered to the subject. The subject's own immunoglobulins can be used for delivery of an antibiotic or cytotoxic moiety, much the same way as the immunoglobulins can be used in implementations related to imaging as disclosed above.

Alternatively, chlamydia infected cells can be selected out of a biological sample from a subject (e.g., blood). Typically, the biological sample is obtained from the subject using sterile technique, and then the chlamydia infected cells are exposed to a molecular construct as described herein that includes an imaging moiety appropriate for FACS or any other fluorescence based sorting technique, or a molecular construct that includes a magnetic nanoparticle. Uninfected and infected cells are then separated using the methods described above. After enrichment for uninfected cells has been performed, the uninfected cells can be transferred back into the subject using sterile technique or frozen for later use.

In some embodiments, the methods include using a mixture of constructs that includes constructs that will bind to more than one type of Fc, e.g., a mix of constructs including FcR binding fragments (or all) of two or more of an IgG, IgA, IgE, and/or IgM. In this way, multiple cell types can be targeted simultaneously.

VIII. Molecular Construct Formulations in Therapeutic and Diagnostic Applications

The molecular constructs described herein can be incorporated into pharmaceutical compositions for use in diagnostic and therapeutic methods. Such compositions typically include the molecular construct and a pharmaceutically acceptable carrier. As used herein the language “pharmaceutically acceptable carrier” includes solvents, dispersion media, coatings, antibacterial and anti-fungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, intravenous, intradermal, subcutaneous, oral, inhalation, transdermal, transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a bind toer such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

In some implementations, the active components are prepared with carriers that will protect the active components against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such pharmaceutical compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any component used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms). Such information can be used to more accurately determine useful doses in humans. Levels in plasma may be measured, for example, by radioimmunoassay to detect the administered polypeptides or antibodies.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered at least one time per week for between about 1 to 10 weeks, preferably between 2 to 8 weeks, more preferably between about 3 to 7 weeks, and even more preferably for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

For antibodies, the preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible.

The disclosed compositions and methods are further illustrated by the following examples. The examples are provided for illustrative purposes only. They are not to be construed as limiting the scope or content of the invention in any way.

### EXAMPLES

### Example 1

### Infection of Human Eosinophils by Chlamydia in vivo

Buffy coat (BC) samples, a mixture of cells including primarily white blood cells, were isolated from fresh human blood samples anti-coagulated with EDTA. Direct smears were made by placing one drop of the BC as well as some red blood cells on a clean glass slide and using a second slide held at a 30-40° angle to complete the smear. The smears were allowed to dry and were then fixed with either heat or 70% methanol (10 minutes at room temperature).

Chlamydial inclusions were detected with a rabbit anti-chlamydia elementary infectious body (EB) antiserum and visualized using an anti-rabbit tetramethylrhodamine isothiocyanate (TRITC) conjugated secondary antibody [red]. Eosinophil peroxidase were detected with a mouse monoclonal antibody and visualized with an anti-moue secondary antibody conjugated with fluorescein isothiocyanate (FITC). Human immunoglobulin was identified with an anti-human Fc monoclonal antibody and visualized with an anti-mouse secondary antibody conjugated with FITC. Optical sections of immunofluorescence throughout the cells were taken using a laser confocal optical system. Images captured at a magnification of 630× using a Bio-Rad MRC-600 Laser Confocal Microscope system. Co-localization of FITC and TRITC staining was determined by merging the respective images for each fluorophore, using the Confocal Assistant™ version 4.02 Image Processing Software.

In optical sections (at 600× magnification) of cells dual immunostained for eosinophil peroxidase and chlamydia, the presence of chlamydial cells in a human eosinophil was clearly observable, demonstrating infection of this type of white blood cell in vivo. A series of images of the same cell stained for human immunoglobulin and clearly demonstrated internalization of human immunoglobulin in the cell, and merged images of chlamydia and human immunoglobulin staining in the cell showed co-localization of the two within the chlamydial inclusion which appeared in orange/yellow.

These results demonstrate that human eosinophils from normal subjects are often infected with Chlamydia in vivo, and IgG is taken up by these cells and sequestered in chlamydial inclusions.

### Example 2

### Colocalization of Chlamydia pneumoniae or Chlamydia trachomatis serovar K with Internalized Immunoglobulin in Cells Infected in vitro

Chlamydial strains: C. pneumoniae AR39 (Cpn), C. caviae, Guinea Pig Inclusion Conjunctivitis (GPIC strain), C. trachomatis serovars A/Har-13, Har-36B, C/TW-3, E/VW-KX, F, K/VR887, mouse pneumonitis agent, (MoPn) and Lymphogranuloma venereum, (LGV 434) were grown in HeLa 229 cells without centrifuge assistance. Infectious EBs were purified by renografin (Squibb diagnostics, New Bronswick, N.J.) density gradient centrifugation. Alternatively, lysates from infected cells were used to infect monolayers.

Cell lines used: McCoy cells (derived from a murine cell line), were obtained from the American Type Culture collection and were grown in minimum essential medium with insulin (IMEMZO, Irvine Scientific, Santa Ana, Calif.) with 5% fetal bovine serum (FBS) (Atlanta Biologicals, Norcross, Ga.). Cells were grown to confluence on 12 mm coverslips in 24 well plates (Becton Dickinson Labware, Franklin Lakes, N.J.). The cells were then infected using lysates of cells that had been previously infected with serovar K of C. trachomatis or C. pneumoniae. A dilution of 1:150 or 1:200 was made using the standard complete cycloheximide overlay media (Bio-Whittaker, Walkersville, Md.) containing 10% FBS, 1× L-glutamine (CCOM, layered onto the coverslip containing monolayers, and incubated for 48-96 hours at 37° C. with 5% CO₂. Coverslips with the cell monolayers were harvested, rinsed with phosphate buffered saline (PBS), fixed with 70% cold methanol, stored and subsequently immunostained following protocols similar to that described in detail previously (Norkin et al., Exp. Cell Res. 266:229-238 (2001) and Stuart et al., Exp. Cell Res. 287:67-78 (2003). Briefly, infected cells were immunostained with a guinea pig anti-chlamydia polyclonal antibody (Biomedia, Foster City, Calif.) and goat anti bovine immunoglobulin G (IgG) (Jackson Immunoresearch, West Grove, Pa.) for 1 hour at 37° C. Following four washes with PBS, the bound antibodies were detected using a 1:50 dilution of TRITC-conjugated goat anti-guinea pig and FITC-conjugated goat anti-bovine secondary antibodies (Jackson Immuno Research, West Grove, Pa.). Following incubation for 1 hour at RT and 4 rinses with (PBS), coverslips were mounted onto slides using Fluoromount-G (Southern Biotechnology Associates Inc., Birmingham, Ala.). Slides were examined at 630× using a Bio-Rad MRC-600 Laser Confocal Microscope system. Images were captured and as relevant, merged using the Confocal Assistant™ version 4.02 Image Processing Software.

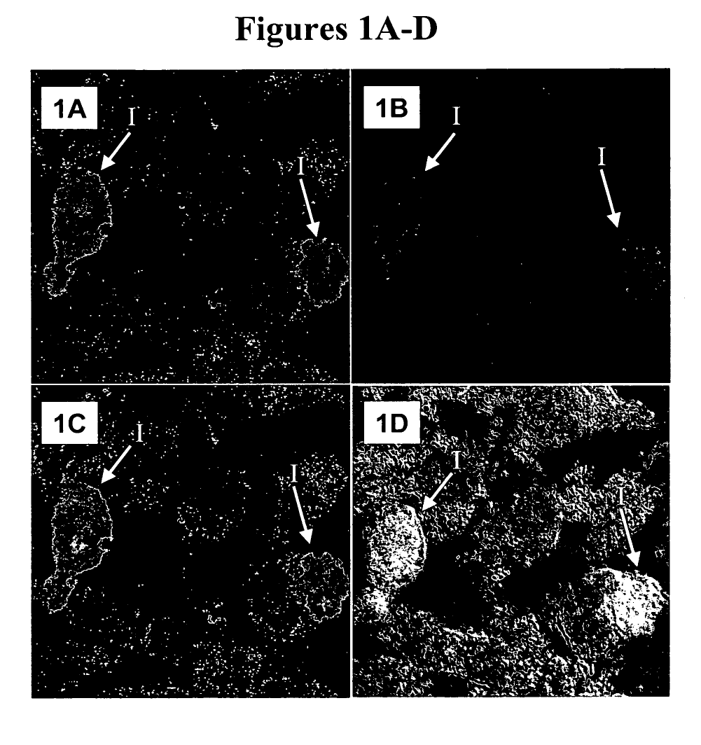

FIG. 1A shows staining of two McCoy cells infected with Chlamydia trachomatis with accumulations of bovine immunoglobulin (Ig) labeled (green in original, indicated with white arrows), surrounded by cells with no Ig staining (note the bright field image of the cells in FIG. 1D). FIG. 1B shows staining of the same cells for chlamydia, labeled in red in the original, identifying chlamydial inclusions. FIG. 1C shows a merged image demonstrating the co-localization of the Ig and chlamydial antigens within the chlamydial inclusion compartment. FIG. 1D is an overlay of the merged image from FIG. 1C with a differential interference contrast (DIC) image of the same field of cells, demonstrating the absence of Ig staining in uninfected cells neighboring the chlamydia infected cells.

An experiment analogous to that demonstrated in FIGS. 1A-D, with the critical difference being that Chlamydia pneumoniae was used to infect the McCoy cells, produced similar results.

These results clearly demonstrate that infection of cells with the two major pathogenic strains of Chlamydia in humans are associated with immunoglobulin internalization and co-localization in chlamydial inclusions.

### Example 3

### “Bulky” FITC Conjugated IgG is Internalized

To determine whether Ig conjugated to a large moiety would be internalized by living cells, FITC conjugated IgG was added to cell culture media of Chlamydia infected J774A.1 macrophages and the next morning samples were rinsed, and fixed (70% MeOH) and immunostained to detect Chlamydia (TRITC-red). Since the internalized IgG was pre-labeled with FITC (green), it already would be visible by confocal fluorescent microscopy.

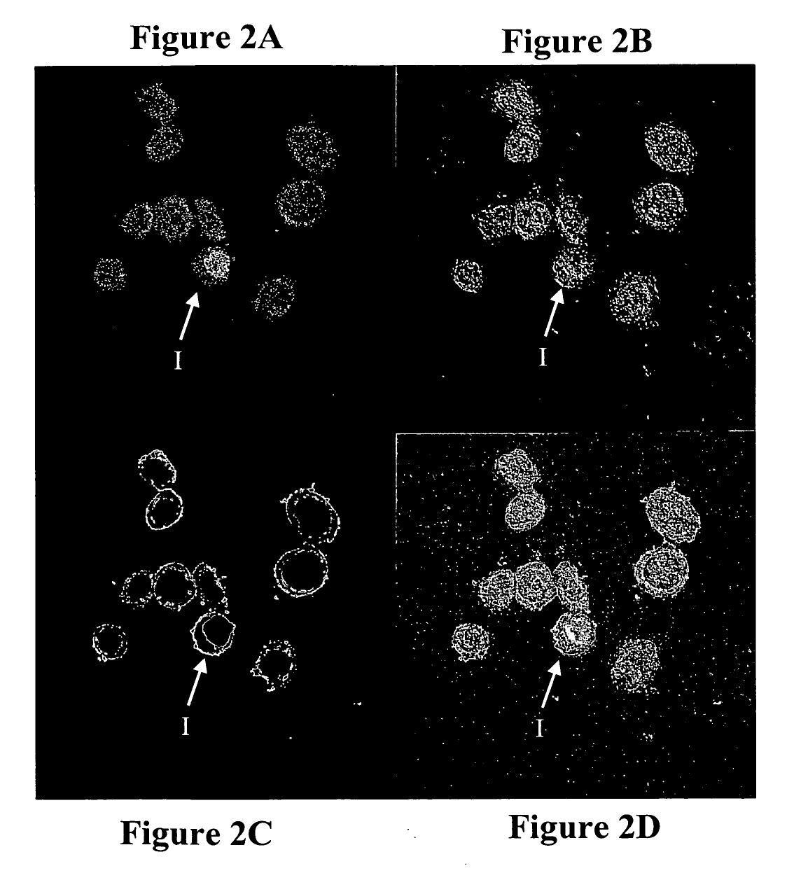

As FIGS. 2A-D show, the FITC labeled IgG does become internalized (2A, infected cell indicated with white arrow), and the merge of FITC and TRITC images (2D) shows the chlamydial antigens and the labeled IgG are in the same optical section.

Therefore this bulkier IgG also is readily internalized and accumulates within the chlamydial inclusion.

In similar experiments, a FITC labeled F(ab′)2 was not internalized, demonstrating that this effect requires the Fc region.

### Example 4

### Immunoglobulin is Sequestered within Chlamydial Inclusions in both the Active Infection State and in the Persistent Infection State

To determine whether Ig is taken into chlamydial inclusions in both active and persistent infections, cultured cells were treated with goat IgG for 30 seconds.

For these in vitro cultures goat IgG was used, since the culture media contains fetal calf serum which contains bovine IgG. Purified goat IgG was added to normal cultures that have active Chlamydia trachomatis infections and separately to cultures in which the Chlamydia trachomatis infection was driven into persistence by treatment with penicillin. Thirty seconds later, the Goat IgG containing media was removed and the samples rinsed and fixed. Samples were immunostained with anti Goat IgG (FITC labeled-green) and also anti-Chlamydia (TRITC labeled-Red), and visualized using medial metaconfocal optical microscopy.

The appearance of yellow in the original images indicated colocalization of the chlamydial antigens and the Goat IgG are present in the same optical section, in both types of infections. These results demonstrate that the Goat IgG is Ig is rapidly taken into chlamydial inclusions in both active and persistent infections.

### Example 5

### JY Cells, from a Human B Cell Line, are Susceptible to Infection with Chlamydia

Chlamydiacae are well known for their ability to disrupt host cell physiology, altering signal transduction, motility, or trafficking of cellular substances (Byrne, (2003) Proc. Natl. Acad. Sci. U.S.A. 100:8040-2). These pathogens differentially traffic cellular components and are able to establish a translocation of host signals, causing a range of activities including upregulation of transporters (Bavoil et al., Microbiology 146(11):2723-31(2000)). Previous research also has demonstrated Chlamydia infected host cells direct the fusion of vesicles derived from the trans-Golgi net-work (TGN). This allows the replicating pathogen access to sphingomyelin as well as sphingolipids and cholesterol (Stuart et al., Exp. Cell. Res. 287:67-78 (2003); Norkin et al., Exp. Cell Res. 266:229-38 (2001); Carabeoet al., Proc. Natl. Acad. Sci. U.S.A. 100:6771-6 (2003)). In addition to the lipid containing components, host cell derived intermediate filament (IF) protein and β-catenin both have been demonstrated to accumulate and co-localize within the inclusions of Chlamydia-infected cells (Prozialeck et al., Infect. Immun. 70:2605-13(2002); Stuart and Brown, Current Microbiology 1992:329-335(1992)).

The demonstrated re-distribution of both IF and β-catenin protein, led us to examine inclusions formed when Ctr serovar K infects a human B cell line, JY, in vitro. The JY cell line is a functional B cell that produces and exports immunoglobulin.

Non-adherent JY and microglial cells were pelleted by centrifugation and resuspended in a solution containing a 1:100 dilution of C. trachomatis serovar K in Cycloheximide Overlay Media (COM) enriched with 10% FBS for 72 hours. Adherent McCoy, J774A.1, and Hec-1B cells were grown to confluency on coverslips in 12 well plates and then had chlamydial lysate solution added. Non-adherent cells were harvested by centrifugation, resuspended and washed in PBS by 30 seconds of centrifugation at 12,400 rpm. Cells were resuspended in 15 μl of fresh PBS and smeared along a glass slide and allowed to air dry. Adherent cells had lysate removed and all cells were fixed with cold 70 % methanol and then immunostained with a 1:100 dilution of a rabbit anti-Chlamydia EB anti-sera primary (made in house) and a 1:200 dilution of Rhodamine (TRITC)-conjugated goat anti-rabbit IgG and either 1:100 FITC conjugated anti-bovine Ig or anti-human Ig (JY cells only) secondary. Other slides had a 1:4 dilution of polyclonal guinea pig anti-Chlamydia (Biomeda, Foster City, Calif.) and a 1:200 rabbit anti-bovine serum albumin (Sigma) primary and a 1:100 TRITC anti-guinea pig Ig with a 1:100 FITC anti-rabbit Ig. Slides were examined and digitally documented using a Zeiss LSM 510 Meta Confocal System.

The results showed that Chlamydia containing regions of infected cells also contain accumulated bovine and human IgG, demonstrating co-localization of the immunoprobes for human and bovine IgG proteins with Chlamydia.

These results indicate that JY, a human B cell line that produces and secretes human IgG, is readily infected with Chlamydia in vitro. Further, infected JY cells take up bovine Ig (blue stain) from the cell culture media, and the Ig localizes to the inclusion.

### Example 6

### Peripheral Blood Cells Immunostained for IgG and Chlamydia

Buffy coat (BC) samples were isolated from EDTA anti-coagulated normal blood donors (NBD) from Baystate Medical Center, Springfield, Mass. Direct smears were made by placing one drop of the buffy coat as well as some RBCs on a clean glass slide and using a second slide held at a 30-40° angle to complete the smear. The smears were allowed to dry and then fixed using 70% methanol. A 1:4 dilution of a polyclonal guinea pig anti-Chlamydia (Biomeda, Foster City, Calif.) and a 1:1000 monoclonal mouse anti-human IgG Fc (Pel-Freez, Rogers, Ariz.) was incubated on the slides in a moist chamber for 1 hour at room temperature (RT). Slides were then rinsed with PBS and a 1:100 dilution of TRITC-conjugated goat anti-guinea pig and either a 1:100 FITC goat human 1 g (H&L) secondary antibody or a 1:100 FITC goat anti-mouse Ig (H+L) added for 1 hour. Slides were examined and digitally documented using a Zeiss LSM 510 Meta Confocal System.

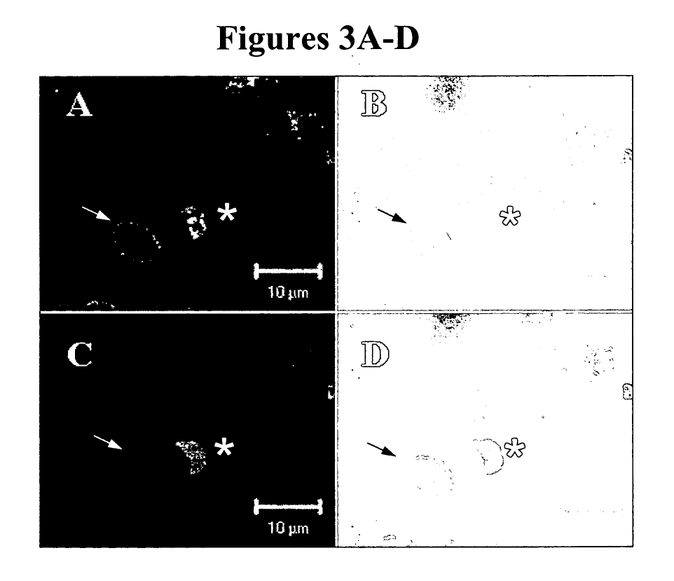

The results are shown in FIGS. 3A-D. In FIG. 3A, anti-human IgG Fc specific mAb binding was detected with a FITC conjugated anti-mouse secondary antibody. The differential interference contrast (DIC) image of the same field, in FIG. 3B, showed that numerous cells are present. FIG. 3C shows an inclusion in an infected cell (indicated by asterisk), detected with a rabbit anti-Chlamydia antibody and a TRITC conjugated anti-rabbit secondary antibody. FIG. 3D, a merge of confocal images in 3A-C, indicates the Chlamydia and IgG immuno-stained materials co-localize in the same optical section. Note that uninfected cells also present in the field show no internalized staining by either antibody although a FITC⁺ rim is evident for some cells in this optical section and indicates human immunoglobulin normally associates only with the surface of uninfected cells. White arrows in 3A and 3C show no Chlamydia in a cell, black arrow in 3B and 3D show NO IgG inside the same cell.

Therefore, in vivo, human peripheral blood cells infected with Chlamydia, but not uninfected cells, take up and accumulate IgG in their chlamydial inclusions. Notably, other cells in the same smear do not bind to the anti-Chlamydia antibody and do not show internalized accumulations of IgG. Therefore in vivo treatment should not result in aberrant IgG internalization. Some background signal, possibly due to binding by cell surface IgG receptors on uninfected cells, can be seen as light ‘outlines’ (green in original).

### Example 7

### Hec-1B Cells Deficient in Monomeric Ig Receptors Show No Internalization of Bovine IgG, but Do Internalize IgA

Hec-1B cells are a human endometrial carcinoma cell line that displays a polymeric Ig Fc receptor (pIg FcR), but does not display a monomeric FcR (mFcR).

Hec-1B cells were infected with C. trachomatis serovar K were fixed at 72 hours post-infection, then dual immunostained to detect Chlamydia and bovine IgG, as described in Example 6.

Immuno-staining Hec-1B cells with a rabbit anti-Chlamydia primary antibody and a TRITC-conjugated secondary anti-rabbit antibody demonstrated the presence of Chlamydia inclusions within cells. Immunostaining with a FITC anti-bovine IgG secondary antibody showed no evidence of bovine IgG within these highly infected Hec-1B cells. The absence of FITC within the cells and the absence of any colocalization verified there was no detectible bovine IgG within the inclusions. DIC images were used to show the cell cluster and the large chlamydial inclusions within the cells.

To determine whether the Hec-1B cells, which display a polymeric Ig Fc receptor (pIg FcR), would internalize polymeric IgA, the experiments were repeated using a human IgA preparation. The IgA is a polymeric antibody. The results indicated that IgA was internalized by Hec-1B cells, and colocalized to Chlamydia inclusions.

These results indicate that Chlamydia infected cells lacking mFcR can be targeted using an antibody that, like IgA, is recognized by a pFcR. Likewise, we theorize that there are cells that express Fc receptors for IgE that could be targeted with an IgE based component.

### Example 8

### Infected J774A.1 Cells do not Internalize Bovine Serum Albumin

To demonstrate the specificity of uptake of Ig, J774A.1 cell monolayers were infected with C. trachomatis serovar K for 72 hours, then fixed and dual immunostained. Chlamydia was detected with a rabbit anti-Chlamydia antibody and a TRITC conjugated secondary antibody; an antibody specific for bovine serum albumin was detected with a FITC-conjugated secondary antibody, as described above in Example 6.

A TRITC secondary antibody was used to detect bound anti-Chlamydia antibody and demonstrate the chlamydial inclusion location. A FITC secondary antibody was used to identify bound anti-bovine serum albumin antibody. It was present on the outer cell membrane rim, but there is no evidence of serum albumin internalized within either of the cells. A merge of the anti-Chlamydia and anti-bovine serum albumin images showed no yellow color. This indicated that there was no co-localization of the two immunoprobes. A DIC image showed granular-appearing inclusions. Black areas seen in the images were sections through nuclear regions which remain unstained. Similar results were obtained with Chlamydia trachomatis serovar B, an ocular serovar.

These results indicate that indeed the internalization of Ig (e.g., IgG or pIgA), is not a non-specific event, but rather a very specific event that is specifically associated with infection by Chlamydia. Both C. trachomatis and C. pneumoniae have been tested for induction of Ig uptake, and the phenomenon occurs for both species of Chlamydia. It also is not restricted to the genital tract-associated Chlamydia trachomatis-serovars D-K and LGV, because the same phenomenon has been observed with the ‘ocular’ serovar B.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of detecting chlamydia in a cell, the method comprising:
(a) contacting the cell with a molecular construct comprising a first portion comprising an amino acid sequence that binds to an Fc receptor, and a second portion comprising a detectable moiety; and
(b) determining the presence of the molecular construct inside the cell, wherein the presence of the polypeptide indicates that the cell is infected with chlamydia.

2. The method of claim 1, wherein the detectable moiety comprises one or more of a nanoparticle, a fluorescent protein, or an enzyme that can yield a detectable reaction product.

3. The method of claim 1, wherein determining the presence of the molecular construct inside the cell comprises acquiring an image of the cell before and after contacting the cell with the polypeptide.

4. The method of claim 1, wherein the cell is obtained from a subject.

5. The method of claim 4, wherein the presence of the molecular construct inside the cell indicates that the subject is infected with Chlamydia.

6. The method of claim 1, wherein the cell is in a living subject, and determining the presence of the molecular construct inside the cell comprises performing an in vivo assay to detect the detectable moiety.

7. The method of claim 6, wherein the assay is magnetic resonance imaging (MRI), positron emission tomography (PET), computed tomography (CT), or ultrasound.

8. A method of separating chlamydia infected cells from uninfected cells, the method comprising:
(a) contacting a population of cells comprising chlamydia infected cells and uninfected cells with a molecular construct comprising a first portion consisting of an amino acid sequence that binds to an Fc receptor, and a second portion comprising a label, under conditions that allow the infected cells to internalize the construct; and
(b) separating the infected cells from the uninfected cells based on the presence of the label in the infected cells.

9. The method of claim 8, wherein the label comprises a magnetic nanoparticle, and separating the cells comprises applying a magnetic field to the population of cells under conditions that allow separation of the cells.

10. The method of claim 8, wherein the label comprises a fluorescent moiety; and separating the cells comprises using fluorescence-based cell sorting.

11. The method of claim 8, wherein the population of cells is obtained from a subject.

12. The method of claim 11, further comprising administering the uninfected cells to the subject.

13. A molecular construct comprising a first Fc receptor (FcR) binding portion consisting of all or a fragment of an immunoglobulin heavy chain that binds to an Fc receptor, and a second portion consisting of a cytotoxic moiety.

14. The molecular construct of claim 13, wherein the fragment is from an IgA, IgQ IgM, or IgE heavy chain.

15. The molecular construct of claim 13, wherein the first portion comprises an Fc region or an scFv with a Fc tail portion.

16. The molecular construct of claim 13, wherein the second portion comprises an intracellular cytotoxin.

17. The molecular construct of claim 13, wherein the cytotoxin is selected from the group consisting of a proapoptotic protein, a polypeptide toxin, and an enzyme that converts a prodrug into a cytotoxic compound.

18. A method of treating a population of cells comprising chlamydia infected cells to reduce the number of infected cells in the population, the method comprising contacting the population of cells with the molecular construct of claim 13.

19. A method of treating a chlamydia infection in a subject, comprising administering to the subject an effective amount of the molecular construct of claim 13.

20. A molecular construct comprising a first Fc receptor (FcR) binding portion consisting of all or a fragment of an immunoglobulin heavy chain that binds to an Fc receptor, linked to a nucleic acid molecule.

21. The molecular construct of claim 20, wherein the nucleic acid molecule comprises a sequence encoding a therapeutic gene product.

22. The molecular construct of claim 20, wherein the nucleic acid molecule comprises an antisense or RNAi construct that targets a chlamydia gene.

23. A method of treating a population of cells comprising chlamydia infected cells to reduce the number of infected cells in the population, the method comprising contacting the population of cells with the molecular construct of claim 20.

24. A method of treating a chlamydia infection in a subject, the method comprising administering to the subject an effective amount of the molecular construct of claim 20.
